(19) United States
(12) Patent Application Publication
(10) Pub. No.: US 2023/0364052 A1
(43) Pub. Date: Nov. 16, 2023

(54) COMPOSITION AND METHOD FOR TREATING AUTISM

(71) Applicant: ZELIRA THERAPEUTICS OPERATIONS PTY LTD, Perth (AU)

(72) Inventor: Harry KARELIS, Perth (AU)

(21) Appl. No.: 18/323,860

(22) Filed: May 25, 2023

Related U.S. Application Data
(62) Division of application No. 16/646,055, filed on Mar. 10, 2020.

(30) Foreign Application Priority Data
Sep. 15, 2017 (AU) 2017903766
Jan. 18, 2018 (AU) 2018900276

(51) Int. Cl. A61K 31/352; A61P 25/00; A61K 9/00; A61K 31/05; A61K 36/185; A61K 47/44

(52) U.S. Cl. CPC A61K 31/352; A61P 25/00; A61K 9/006; A61K 31/05; A61K 36/185; A61K 47/44; A61K 2236/333

(57) ABSTRACT

The invention relates to pharmaceutical compositions comprising cannabidiol (CBD) and Δ⁹-tetrahydrocannabinol (THC), and their use in the treatment of autism. The invention also relates to methods for treating autism.

### FIELD

The invention relates to pharmaceutical compositions comprising cannabidiol (CBD) and Δ⁹-tetrahydrocannabinol (THC), and their use in the treatment of autism. The invention also relates to methods for treating autism.

### BACKGROUND

The biological activity of Cannabis is well known, and has led it to become a “recreational” drug. However, with the discovery of a class of cannabinoid (CB) receptors, and the relaxation of laws regulating Cannabis use—in some jurisdictions, decriminalisation—there now exists the opportunity to explore the potential of Cannabis as a source of new therapeutics.

According to the American Psychiatric Association, autism is a complex developmental disorder that can cause problems with thinking, feeling, language and the ability to relate to others. It is a neurological disorder, which means it affects the functioning of the brain. The effects of autism and the severity of symptoms are different in each person. It is often diagnosed in childhood and is typically a life-long condition.

The current approved therapeutics for treating autism are risperidone and/or aripiprazole. However, neither of these therapies is effective at improving the core symptoms of autism for a vast majority of patients and have been associated with frequent adverse events.

There is a growing movement of patients suffering from diseases, such as autism, to seek natural remedies as alternative or complementary therapy.

Accordingly, there is a continuing need to develop new treatments for autism, which are based, at least in part, on a natural source. It would be advantageous to develop new treatments requiring minimal dosing of active constituents for administration to patients.

### SUMMARY

The inventors believe that treatment of autism patients with a pharmaceutical composition comprising CBD and THC can improve the quality of life of the patients themselves as well as their families and carers. The therapy may lead to the treatment of symptoms of autism or symptoms associated with autism in the patients as assessed by the CGI-Improvement score. The therapy may provide preferable treatment of these symptoms compared with that provided by conventional therapies, such as risperidone or aripiprazole treatment. Further, the therapy may prevent or reduce the adverse events and side-effects associated with conventional therapies, so that the therapy may be better tolerated than conventional therapies, such as risperidone or aripiprazole treatment.

In one aspect, there is provided a pharmaceutical composition comprising CBD and THC in a ratio of CBD:THC from about 0.1:5 to about 5:0.1 and optionally one or more pharmaceutically acceptable excipient(s).

In another aspect, there is provided a method for treating autism comprising administering to a patient in need thereof an effective amount of a pharmaceutical composition of the invention.

In a further aspect, there is provided a method for treating a symptom of autism or a symptom associated with autism selected from reduced sociability, tantrums, poor use of language, repetitive behaviour, self-injurious behaviour, irritability, hyperactivity, poor ability to focus, unexplained weight loss, fever, fatigue, pain, and skin changes or a combination thereof comprising administering to a patient in need thereof an effective amount of a pharmaceutical composition of the invention.

In still a further aspect, there is provided use of CBD and THC in a ratio of CBD:THC from about 0.1:5 to about 5:0.1 in the manufacture of a medicament for treating autism.

In yet another aspect, there is provided use of CBD and THC in a ratio of CBD:THC from about 0.1:5 to about 5:0.1 in the manufacture of a medicament for treating a symptom of autism or a symptom associated with autism selected from reduced sociability, tantrums, poor use of language, repetitive behaviour, self-injurious behaviour, irritability, hyperactivity, poor ability to focus, unexplained weight loss, fever, fatigue, pain, and skin changes or a combination thereof.

In another aspect, there is provided a pharmaceutical composition comprising CBD and THC in a ratio of CBD:THC from about 0.1:5 to about 5:0.1 for treating autism.

In a further aspect, there is provided a pharmaceutical composition comprising CBD and THC in a ratio of CBD:THC from about 0.1:5 to about 5:0.1 for treating a symptom of autism or a symptom associated with autism selected from reduced sociability, tantrums, poor use of language, repetitive behaviour, self-injurious behaviour, irritability, hyperactivity, poor ability to focus, unexplained weight loss, fever, fatigue, pain, and skin changes or a combination thereof.

In yet another aspect, there is provided a kit comprising in separate parts: (a) CBD, and (b) THC, wherein the amount of CBD in part (a) and the amount of THC in part (b) are in a ratio of CBD:THC from about 0.1:5 to about 5:0.1.

In still another aspect, there is provided an agent for treating autism comprising CBD and THC in a ratio of CBD:THC from about 0.1:5 to about 5:0.1.

### BRIEF DESCRIPTION OF DRAWINGS

The present application will be further described, by way of example only, with reference to the accompanying drawings, in which:

FIG. 1 shows a graphic outlining interactions of the CB₁ and CB₂ receptors as part of the human endocannabinoid system.

FIG. 2 shows a graphic of the human brain indicating regions of high and moderate CB₁ receptor expression. Regions with high CB₁ expression include the cerebral cortex, cerebellum, hippocampus, basal ganglia and prefrontal cortex. Regions with moderate CB₁ expression include hypothalamus, periaqueductal gray, nucleus of the solitary tract, spinal cord, brain stem and amygdala.

FIG. 3 shows a pie chart showing the initial severity of ASD in the patients included in the study of Example 2.

FIG. 4 shows a chart of CGI-I scores post-treatment in the patients included in the study of Example 2.

### DESCRIPTION OF EMBODIMENT(S)

Before describing the present invention in detail, it is to be understood that this invention is not limited to particularly exemplified pharmaceutical compositions, methods of production or treatment, which may, of course, vary. It is also to be understood that the terminology used herein is for the purpose of describing particular embodiments of the invention only, and is not intended to be limiting.

The inventions described and claimed herein have many attributes and embodiments including, but not limited to, those set forth or described or referenced in this summary section, which is not intended to be all-inclusive. The inventions described and claimed herein are not limited to or by the features or embodiments identified in this summary section, which is included for purposes of overview illustration only and not limitation.

All publications, patents and patent applications cited herein, whether supra or infra, are hereby incorporated by reference in their entirety. However, publications mentioned herein are cited for the purpose of describing and disclosing the protocols and reagents which are reported in the publications and which might be used in connection with the invention. Nothing herein is to be construed as an admission that the invention is not entitled to antedate such disclosure by virtue of prior invention.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### Pharmaceutical Compositions

The present invention provides a pharmaceutical composition comprising CBD and THC.

The pharmaceutical composition comprises CBD and THC in ratio of CBD:THC from about 0.1:5 to about 5:0.1. For example, the pharmaceutical composition may comprise CBD and THC in a ratio of CBD:THC from about 0.1:4 to about 4:0.1, about 0.15:3.5 to about 3.5:0.15, about 0.2:3 to about 3:0.2, about 0.4:3 to about 3:0.4, or about 0.45:1 to about 2.6:1. In some embodiments, the pharmaceutical composition may comprise CBD and THC in a ratio of CBD:THC of about 0.3:1, about 0.4:1, about 0.45:1, about 0.5:1, about 0.7:1, about 0.75:1, about 0.8:1, about 0.8:1, about 1:1, about 1.1:1 about 1.2:1, about 1.3:1, about 1.4:1, about 1.5:1, about 1.6:1, about 1.66:1, about 1.7:1, about 1.8:1, about 1.9:1, about 2:1, about 2.1:1, about 2.2:1, about 2.3:1, about 2.4:1, about 2.5:1, about 2.6:1, about 2.7:1, about 2.8:1, about 2.9:1 or about 3:1. In one embodiment, the pharmaceutical composition comprises CBD and THC in a ratio of CBD:THC of about 1:1. The ratio of CBD:THC may be represented as a single numeral, for example, a ratio of CBD:THC of 2:1 is equivalent to a ratio of CBD:THC of 2, and ratio of CBD:THC of 1:2 is equivalent to a ratio of CBD:THC of 0.5. In some embodiments, the ratio of CBD:THC is about 5, for example, the ratio of CBD:THC may be about 4.5, about 4, about 3.5, about 3, about 2.7, about 2.6, about 2.5, about 2.53, about 2.4, about 2.3, about 2.2, about 2.1, about 2, about 1.9, about 1.8, about 1.7, about 1.66, about 1.6, about 1.5, about 1.4, about 1.3, about 1.2, about 1.1, about 1, about 0.9, about 0.8, about 0.7, about 0.6, about 0.5, about 0.45, about 0.4, about 0.3, about 0.2 or about 0.1. The ratio of CBD:THC may be between any of these values, for example, from about 5 to about 0.1, from about 0.1 to about 5, from about 3 to about 0.2, from about 2.6 to about 0.4, or from about 1.53 to about 0.45.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

The ratio of CBD to THC may be readily determined by methods known in the art, including High-Performance Liquid Chromatography (HPLC).

The amount of CBD and THC included in the pharmaceutical composition will depend on a number of factors, including the other components, the subject's characteristics (e.g. size, species, sex, etc.), and the severity of the disease to be treated.

In some embodiments, the amount of THC is greater than the amount of CBD in the pharmaceutical composition. In these embodiments, the ratio of CBD:THC is less than 1, for example, the ratio of CBD:THC expressed as a single numeral may be less than or equal to about 0.9, about 0.8, about 0.7, about 0.6, about 0.5, about 0.45, about 0.4, about 0.3, about 0.2 or about 0.1. The ratio of THC:CBD may be at least about 1.1:1, about 1.5:1, about 1.8:1, about 1.9:1, about 2:1, about 2.1:1, about 2.2:1, about 2.3:1, about 2.4:1, about 2.5:1, about 3:1, about 3.5:1 or higher. For example, ratio of THC:CBD may be from about 1.1:1 to about 3.5:1 or about 2:1 to about 2.4:1.

In other embodiments, the amount of CBD is greater than the amount of THC in the pharmaceutical composition. In these embodiments, the ratio of CBD:THC is greater than 1, for example, the ratio of CBD:THC expressed as a single numeral may be greater than or equal to about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 3.5, about 4, about 4.5, or about 5. The ratio of CBD:THC may be between any of these ratios, for example, may be from about 1.1 to about 5, about 1.2 to about 2.7, about 1.2 to about 1.6, or about 2.3 to about 2.7.

In some embodiments, the amounts of CBD and THC are balanced. In such embodiments, the ratio of CBD:THC may be from 1:2 to 2:1. For example, a balanced amount of CBD and THC may be expressed as a ratio of CBD:THC as a single numeral and be from 0.5 to 2, 0.5 to 1.6, 0.7 to 1.6 or 0.8 to 1.6. In still other embodiments, the amounts of CBD and THC are substantially the same in the pharmaceutical composition. Substantially the same amounts of CBD and THC may mean that the amount of each cannabinoid is present within 5 wt % of the other in the pharmaceutical composition.

In some embodiments, the pharmaceutical composition comprises CBD in a minimum amount of at least about 0.001 wt %, for example, at least about 0.005 wt %, about 0.01 wt %, or about 0.02 wt %. In some embodiments, the pharmaceutical composition comprises CBD in a maximum amount of up to about 15 wt %, for example, up to about 10 wt %, about 9 wt %, about 8 wt %, about 7 wt %, about 5 wt %, about 2.5 wt %, about 2 wt %, about 1 wt %, or about 0.5 wt %. In some embodiments where the pharmaceutical composition is a liquid, the pharmaceutical composition comprises CBD in a minimum amount of at least about 0.001 mg/ml, for example, at least about 0.005 mg/ml, about 0.01 mg/ml or about 0.02 mg/ml. In some embodiments where the pharmaceutical composition is a liquid, the pharmaceutical composition comprises CBD in a maximum amount of up to about 150 mg/ml, for example, about 125 mg/ml, about 100 mg/ml, about 90 mg/ml, about 80 mg/ml or about 70 mg/ml. It will be appreciated that the amount of CBD may be within the range from any of these minimum amounts to any of these maximum amounts whether expressed as a percentage by weight or as a concentration in milligrams per millilitre of a liquid composition. All combinations of these minimum and maximum amounts are contemplated. For example, in some embodiments, the pharmaceutical composition comprises CBD in an amount of from about 0.001 wt % to about 80 wt % or from about 0.001 mg/ml to about 150 mg/ml.

In some embodiments, the pharmaceutical composition comprises THC in a minimum amount of at least about 0.001 wt %, for example, at least about 0.005 wt %, about 0.01 wt % or about 0.02 wt. %. In some embodiments, the pharmaceutical composition comprises THC in a maximum amount of up to about 15 wt %, about 10 wt %, about 5 wt %, about 2.5 wt %, about 2 wt %, about 1.5 wt %, about 1 wt %, about 0.5 wt %, about 0.25 wt % or about 0.1 wt %. In some embodiments where the pharmaceutical composition is a liquid, the pharmaceutical composition comprises THC in a minimum amount of at least about 0.001 mg/ml, for example, at least about 0.005 mg/ml, about 0.01 mg/ml or about 0.02 mg/ml. In some embodiments where the pharmaceutical composition is a liquid, the pharmaceutical composition comprises THC in a maximum amount of up to about 125 mg/ml, for example, about 100 mg/ml, about 90 mg/ml, about 80 mg/ml, about 70 mg/ml, about 60 mg/ml or about 50 mg/ml. It will be appreciated that the amount of THC may be within the range from any of these minimum amounts to any of these maximum amounts whether expressed as a percentage by weight or as a concentration in milligrams per millilitre of a liquid composition. All combinations of these minimum and maximum amounts are contemplated. For example, in some embodiments, the pharmaceutical composition comprises THC in an amount of from about 0.001 wt % to about 80 wt % or from about 0.001 mg/mi to about 125 mg/ml.

In some embodiments, the pharmaceutical composition comprises CBD and THC in a minimum total amount of at least about 0.001 wt %, for example, at least about 0.005 wt %, about 0.01 wt % or about 0.05 wt %. In some embodiments, the pharmaceutical composition comprises CBD and THC in a total maximum amount of up to about 25 wt %, for example, up to about 20 wt %, about 15 wt %, about 12.5 wt %, about 11 wt %, about 10 wt %, about 5 wt %, about 2.5 wt %, about 1 wt %, about 0.5 wt %, or about 0.25 wt %. It will be appreciated that the total amount of CBD and THC may be within the range from any of these minimum amounts to any of these maximum amounts. All combinations of these minimum and maximum amounts are contemplated. For example, in some embodiments, the pharmaceutical composition comprises CBD and THC in an amount of from about 0.001 wt % to about 0.25 wt %.

References to CBD and THC (and any other natural product, including cannabinoid(s)) used herein include the relevant compound and pharmaceutically acceptable salts and/or solvates (including hydrates) thereof.

The CBD and THC may be combined from purified forms of the compounds, which may be purified after extraction from a natural source, or produced synthetically or semi-synthetically. Any means known in the art for providing CBD and/or THC is contemplated. In some embodiments, the pharmaceutical composition may comprise a Cannabis extract comprising the CBD and the THC.

THC and CBD do not occur in significant concentrations in Cannabis plant material and are formed during the extraction process through decarboxylation of corresponding carboxylic acid derivatives of these cannabinoids (or cannabinoid acids), which are biosynthesised by the Cannabis plant. The precise concentration of neutral THC or CBD in a Cannabis plant is difficult to quantify due to the potential for decarboxylation of the corresponding cannabinoid acids during analysis. Accordingly, when the pharmaceutical compositions of the invention comprise THC or CBD derived from a natural source, the composition comprises decarboxylated THC and/or CBD.

The extraction process typically comprises a decarboxylation step. Decarboxylation refers to the loss of a carboxyl group during conversion of a carboxylic acid derivative of a cannabinoid into the cannabinoid itself. Δ⁹-Tetrahydrocannabinolic acid (THC-A) and cannabidiolic acid (CBD-A) are not thermally stable and may be decarboxylated by exposure to light or heat. Some studies have also shown that THC-A and CBD-A can be decarboxylated upon exposure to cofactors or certain solvents. Typically, decarboxylation is carried out by heating the extract in the presence of extractant to a temperature of at least 60° C. (e.g. at least 80° C.). This heating step may be maintained for 30 minutes or longer. In some embodiments, the decarboxylation occurs during extraction and/or extractant removal. In some embodiments, decarboxylation occurs during drying of the plant material.

In addition, THC has been shown to oxidise to cannabinol (CBN) when exposed to oxygen and light, including during decarboxylation. Accordingly, in some embodiments, the extraction comprises exposing the extract to light under an oxygen atmosphere. In such embodiments, the pharmaceutical composition will typically further comprise CBN. In other embodiments, the extraction is carried out in the absence of oxygen, for example under an atmosphere of nitrogen.

In embodiments of the invention comprising a Cannabis extract, the pharmaceutical composition may further comprise one or more secondary metabolites. Cannabis plants produce a diverse array of secondary metabolites, including cannabinoids, terpenes and terpenoids, sterols, triglycerides, alkanes, squalenes, tocopherols, carotenoids and alkaloids. The mix of these secondary metabolites varies depending on several factors, including Cannabis variety, part of the Cannabis plant extracted, method of extraction, processing of the extract, and season.

There are several varieties of Cannabis plant, which have been described under two distinct naming conventions. One of these conventions identifies three distinct species of Cannabis plant, namely Cannabis sativa Linnaeus, Cannabis indica LAM., and Cannabis ruderalis. Another convention identifies all Cannabis plants as belonging to the Cannabis sativa L. species, with the various varieties divided amongst several subspecies, including: Cannabis sativa ssp. sativa and ssp. indica. As used herein, the term “Cannabis” refers to any and all of these plant varieties.

Extracts of Cannabis may be prepared by any means known in the art. The extracts may be formed from any part of the Cannabis plant containing cannabinoid, terpene and terpenoid compounds. Extracts may be formed from a leaf, seed, trichome, flower, keif, shake, bud, stem or a combination thereof. The part of the Cannabis plant may be used fresh or dried prior to extraction. All known means of drying the plant material are contemplated. In some embodiments, the extract is formed by contacting any part of the Cannabis plant with an extractant. Any suitable extractant known in the art may be used, including, for example, alcohols (e.g. methanol, ethanol, propanol, butanol, propylene glycol etc.), water, hydrocarbons (e.g. butane, hexane, etc.), oils (e.g. olive oil, vegetable oil, essential oil, etc.), a polar organic solvent (e.g. ethyl acetate, polyethylene glycol, etc.) or a supercritical fluid (e.g. liquid CO2). The extractant may be completely or partially removed prior to incorporation of the Cannabis extract into the pharmaceutical composition, or it may be included in the pharmaceutical composition as a carrier. The extractant may be removed by heating the extract optionally under reduced pressure (e.g. under vacuum). It will be appreciated that some of the more volatile plant metabolites (such as terpenes) may also be removed with the extractant. Accordingly, in some embodiments, removing the extractant may enrich the cannabinoid fraction of the extract.

In some embodiments, the extract is filtered to remove particulate material, for example, by passing the extract through filter paper or a fine sieve (e.g. a sieve with pore sizes of 5 μm). The Cannabis composition may comprise up to about 5% by weight (e.g., up to about 2% by weight) visible particles.

In some embodiments, the Cannabis extract is formed by applying heat and/or pressure to the plant material. Typically, in these embodiments, no extractant is required.

In some embodiments, the Cannabis extract is a Cannabis oil. As used herein, “Cannabis oil” is an extract formed by contacting at least a part of a Cannabis plant with an oil. The extracting oil may optionally be removed. Extracting oils may be selected from olive oil, hemp oil, sesame oil, coconut oil, vegetable oil, canola oil, grape seed oil, almond oil, medium-chain triglyceride (MCT) oil, and any other edible oil, or a combination thereof.

In some embodiments, the Cannabis extract is macerated oil. Any suitable maceration process known in the art may be used. Maceration typically requires contacting plant material with an extractant for a period of time. The use of any extractant described herein is contemplated. The maceration may be conducted at high temperature (e.g. greater than 50° C.), ambient temperature (e.g. about 20-25° C.) or at cold temperature (e.g. less than about 5° C.).

In some embodiments, the Cannabis extract is a resin. Cannabis resin is typically obtained by separating and compressing a Cannabis flower or a part thereof, such as the resin glands, or trichomes. Any method known in the art for preparing the resin is contemplated. For example, the resin may be prepared by contacting the Cannabis flower or part thereof with an extractant (e.g. an alcohol, such as ethanol), filtering the extract and heating the filtrate (e.g. at about 90° C.) to evaporate the extractant.

In some embodiments, the Cannabis extract is an extract formed by contacting an alcohol with Cannabis plant material. The alcohol may be selected from methanol, ethanol, propanol, butanol and combinations thereof.

It will be appreciated that the identity and proportions of compounds extracted from a Cannabis plant material will vary depending on the extractant used and the conditions employed for the extraction. For example, use of a relatively low boiling point extractant, such as methanol or ethanol, may more readily be removed while retaining higher concentrations of other volatile components of the extract, such as terpenes and/or terpenoids. Thus, the lower the boiling point of the extractant selected may provide extracts with higher concentration of volatiles (such as terpenes and/or terpenoids) depending on the extractant removal technique employed.

In some embodiments, one or more additional compounds (e.g. cannabinoid, terpene or terpenoid compounds) may be added to the Cannabis extract. The addition of compounds may be to compensate for natural variations in the relative amounts of certain compounds being expressed in the Cannabis plant. The added compounds may be synthetic versions of the desired compounds, they may be purified compounds obtained from other Cannabis extracts, or they may be added by blending two or more Cannabis extracts.

The term “cannabinoid” as used herein relates to any molecule that has been isolated from a Cannabis plant or synthetically created to have activity involving the endocannabinoid system. The term is used to describe the relevant molecule itself irrespective of its source.

The term “cannabinoid fraction” is used to describe the combination of cannabinoid compounds present in the Cannabis extract.

The term “terpenes” or “terpenoids” as used herein refers to a class of hydrocarbon molecules, which often provide a unique smell. Terpenes are derived from units of isoprene, which has the molecular formula C₅H₈. The basic molecular formula of terpenes are multiples of the isoprene unit, i.e. (C₅H₈)ₙ, where n is the number of linked isoprene units. Terpenoids are terpene compounds that have been further metabolised in the plant, typically through an oxidative process, and therefore usually contain at least one oxygen atom.

The term “terpene fraction” is used to describe the combination of terpene and terpenoid compounds present in the Cannabis extract.

The cannabinoid fraction typically accounts for the majority of the compounds present in the Cannabis extract.

In some embodiments, the Cannabis extract may comprise about 35% to about 95% by weight cannabinoids, for example, about 40% to about 90%, about 45% to about 70% or about 45% to about 55% by weight of the Cannabis extract. In some embodiments, the Cannabis extract comprises about 5% to about 65% by weight of non-cannabinoids, for example, about 5% to about 50%, about 10% to about 40% by weight or about 15% to about 30% by weight non-cannabinoids.

To date, over 100 cannabinoids have been identified in Cannabis extracts. A comprehensive list of these cannabinoids may be found in Mahmoud A. El Sohly and Waseem Gul, “Constituents of Cannabis Sativa.” In Handbook of Cannabis Roger Pertwee (Ed.) Oxford University Press (2014) (ISBN: 9780199662685). Cannabinoids that have been identified in Cannabis plants include: Cannabigerol (E)-CBG-C5, Cannabigerol monomethyl ether (E)-CBGM-C5 A, Cannabigerolic acid A (Z)-CBGA-C5 A, Cannabigerovarin (E)-CBGV-C3, Cannabigerolic acid A (E)-CBGA-C5 A, Cannabigerolic acid A monomethyl ether (E)CBGAM-C5 A and Cannabigerovarinic acid A (E)-CBGVAC3A; (±)-Cannabichromene CBC-C5, (±)-Cannabichromenic acid A CBCA-C5 A, (±)-Cannabivarichromene, (±)-Cannabichromevarin CBCV-C3, (±)-Cannabichromevarinic acid A CBCVA-C3 A; (−)-Cannabidiol CBD-C5, Cannabidiol momomethyl ether CBDMC5, Cannabidiol-C4 CBD-C4, (−)-Cannabidivarin CBDVC3, Cannabidiorcol CBD-CI, Cannabidiolic acid CBDA-C5, Cannabidivarinic acid CBDVA-C3; Cannabinodiol CBNDC5, Cannabinodivarin CBND-C3; Δ⁹-Tetrahydrocannabinol Δ⁹-THC-C5, Δ⁹-Tetrahydrocannabinol-C4 Δ⁹-THCC4, Δ⁹-Tetrahydrocannabivarin Δ⁹-THCV-C3, Δ⁹-Tetrahydrocannabiorcol Δ⁹-THCO-CI, Δ⁹-Tetrahydrocannabinolic acid A Δ⁹-THCA-C5 A, Δ⁹-Tetrahydrocannabinolic acid B Δ⁹-THCA-C5 B, Δ⁹-Tetrahydrocannabinolic acid-C4 A and/or B Δ⁹-THCA-C4 A and/or B, Δ⁹-Tetrahydro-cannabivarinic acid A Δ⁹-THCVA-C3 A, Δ⁹-Tetrahydrocannabiorcolic acid A and/or B Δ⁹-THCOA-CI A and/or B), (−)-Δ⁸-trans-(6aR,10aR)-Δ⁸-Tetrahydrocannabinol Δ⁸-THC-C5, (−)-Δ⁸-trans-(6aR,10aR)-Tetrahydrocannabinolic acid A Δ⁸-THCA-C5 A, (−)-(6aS,10aR)-Δ⁹-Tetrahydrocannabinol (−)-cis-Δ⁹-THC-C5; Cannabinol CBN-C5, Cannabinol-C4 CBN-C4, Cannabivarin CBN-C3, Cannabinol C2 CBN-C2, Cannabiorcol CBN-CI, Cannabinolic acid A CBNA-C5 A, Cannabinol methyl ether CBNM-C5, (−)-(9R,10R)-trans-Cannabitriol (−)-trans-CBT-C5, (+)-(9S,10S)-Cannabitriol (+)-trans-CBT-C5, (±)-(9R,10S/9S,10R)−); Cannabitriol (±)-cis-CBT-C5, (−)-(9R,10R)-trans-10-O-Ethyl-cannabitriol (−)-trans-CBT-OEt-C5, (±)-(9R,10R/9S,10S)-Cannabitriol-C3 (±)-trans-CBT-C3, 8,9-Dihydroxy-Δ6a(10a)-tetrahydrocannabinol 8,9-Di-OH-CBT-C5, Cannabidiolic acid A cannabitriol ester CBDA-C5 9-OH-CBT-C5 ester, (−)-(6aR,9S,10S,10aR)-9,10-Dihydroxyhexahydrocannabinol, Cannabiripsol, Cannabiripsol-C5, (−)-6a,7,10a-Trihydroxy-Δ⁹-tetrahydrocannabinol (−)-Cannabitetrol, 10-Oxo-Δ6a(10a)tetrahydrocannabinol OTHC); (5aS,6S,9R,9aR)-Cannabielsoin CBE-C5, (5aS,6S,9R,9aR)-C3-Cannabielsoin CBE-C3, (5aS,6S,9R,9aR)-Cannabielsoic acid A CBEA-C5 A, (5aS,6S,9R,9aR)-Cannabielsoic acid B CBEA-C5 B; (5aS,6S,9R,9aR)-C3-Cannabielsoic acid B CBEA-C3 B, Cannabiglendol-C3 OH-iso-HHCV-C3, Dehydrocannabifuran DCBF-C5, Cannabifuran CBF-C5), (−)-Δ⁷-trans-(1R,3R,6R)-Isotetrahydrocannabinol, (±)-Δ⁷-1,2-cis-(1R,3R,6S/1S,3S,6R)-Isotetrahydrocannabivarin, (−)-Δ⁷-trans-(1R,3R,6R)-Isotetrahydrocannabivarin; (±)-(IaS,3aR,8bR,8cR)-Cannabicyclol CBL-C5, (±)-(1aS,3aR,8bR,8cR)-Cannabicyclolic acid A CBLA-C5 A, (±)-(IaS,3aR,8bR,8cR)-Cannabicyclovarin CBLV-C3; Cannabicitran CBTC5; Cannabichromanone CBCN-C5, CannabichromanoneC3 CBCN-C3, and Cannabicoumaronone CBCON-C5.

The cannabinoid fraction may comprise a primary (or main) cannabinoid. As used herein, the term “primary cannabinoid” relates to the cannabinoid present in a Cannabis extract is the highest concentration. Typically, the primary cannabinoid may be Δ⁹-Tetrahydrocannabinol (THC) or cannabidiol (CBD). The primary cannabinoid may be present in the Cannabis extract in an amount of at least about 0.1%, about 0.5%, about 1%, about 1.5%, about 2%, about 2.5%, about 3%, about 3.5%, about 4%, about 20%, about 25%, about 30%, about 35%, about 40%, about 45%, about 50% or about 55% by weight of the Cannabis extract. Accordingly, when THC is the primary cannabinoid, the Cannabis extract may comprise at least about 0.1%, about 0.5%, about 1%, about 1.5%, about 2%, about 2.5%, about 3%, about 3.5%, about 4%, about 4.5%, about 20%, about 25%, about 30%, about 35%, about 40% about 45%, about 50% or about 55% by weight Δ⁹-tetrahydrocannabinol (THC), for example, about 0.1% to about 97%, about 0.1% to about 20%, or about 50 to about 90% by weight of Δ⁹-tetrahydrocannabinol (THC). When CBD is the primary cannabinoid, the Cannabis extract may comprise at least about 0.1%, about 0.5%, about 1%, about 1.5%, about 2%, about 2.5%, about 3%, about 3.5%, about 4%, about 20%, about 25%, about 30%, about 35%, about 40%, about 45%, about 50%, about 55% or about 60% by weight CBD, for example, about 0.1% to about 97%, about 0.1% to about 10% or about 50 to about 90% by weight of CBD.

In addition to the primary cannabinoid, the Cannabis extract may further comprise a secondary cannabinoid. As used herein, the term “secondary cannabinoid” relates to the cannabinoid present in a Cannabis extract is the second highest concentration. The secondary cannabinoid is therefore present in the Cannabis extract in an amount less than the primary cannabinoid. In some embodiments where the primary cannabinoid is THC, the secondary cannabinoid may be CBD. In some embodiments where the primary cannabinoid is CBD, the secondary cannabinoid may be THC. The secondary cannabinoid may be present in the Cannabis extract in an amount of at least about 0.001% by weight, for example, at least about 0.005%, 0.01%, 0.05%, 0.1%, 0.5%, 1%, 1.5% or 2% by weight of the extract. The secondary cannabinoid may be present in a maximum amount of less than the amount of the primary cannabinoid, such as up to about 25%, for example, up to about 10%, 9%, 8%, 7%, 6%, 5% by weight of the extract. It will be appreciated that the amount of secondary cannabinoid may be within the range from any of these minimum amounts to any of these maximum amounts

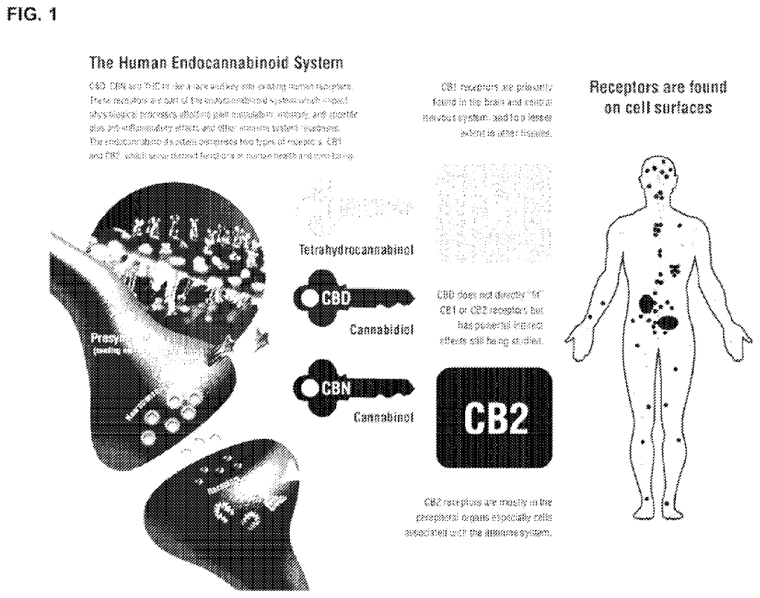

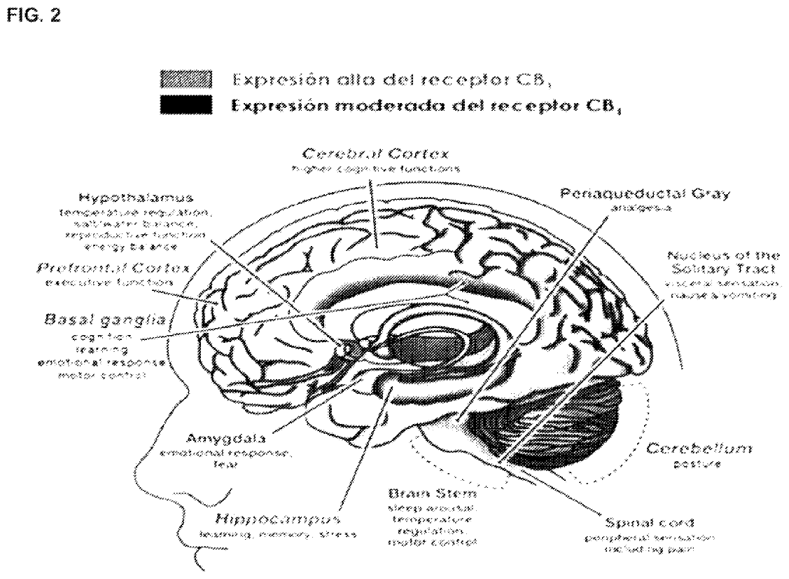

In some embodiments, the Cannabis extract is enriched in one or the other of CBD or THC. It has been shown that endocannabinoids (i.e. naturally occurring cannabinoids), including CBD and THC, interact with a class of G protein-coupled receptors (GPCRs) named the “cannabinoid receptors”, e.g. the CB1 or CB2 receptors (see FIG. 1). However, structurally related cannabinoid compounds may have vastly different activity. Despite these differences in activity, the present invention relies on the activity of CBD and THC in combination.

In some embodiments, the Cannabis extract may comprise at least about 0.001% by weight CBD and/or THC, for example, from about 0.001% to about 99.999% by weight THC and/or CBD, at least about 0.001% to about 20% by weight THC and/or CBD, about 0.01% to about 20% by weight THC and/or CBD, about 0.01% to about 15% by weight THC and/or CBD.

In some embodiments, the Cannabis extract may comprise CBD and THC in a combined weight of at least about 1% by weight, for example, at least about 5% by weight. In some embodiments, the combined amount of CBD and THC may be 1-20%, 1-15%, 6-11% or 50-90% by weight of the pharmaceutical composition. The ratio of THC to CBD may be from about 100:0 to about 0:100, about 100:1 to about 1:100, about 80:1 to about 1:80, about 60:1 to about 1:60, about 40:1 to about 1:40, about 20:1 to about 1:20, about 10:1 to about 1:10, about 5:1 to about 1:5, about 4.5:1 to about 1:4.5, about 4:1 to about 1:4, about 3.5:1 to about 1:3.5, about 3:1 to about 1:3. In some embodiments, the ratio of THC to CBD may be balanced, for example in a ratio of THC:CBD of about 2:1 to about 1:2 or about 1:1. The ratio of THC:CBD may be expressed as a single number by dividing the amount of THC by the amount of CBD present. Accordingly, the ratio of THC:CBD in the pharmaceutical compositions may be 0.001, 0.1, 0.2, 0.3, 0.4, 0.45, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5 or higher. In some embodiments, the ratio of THC:CBD may be between any of these values, for example, from 0.001 to 5, 0.1 to 5, 0.001 to 4, 0.1 to 4, 0.001 to 3, 0.2 to 3 or 0.4 to 2.6.

Typically, the Cannabis extract may also comprise other cannabinoids in addition to CBD and/or THC. These cannabinoids include Δ⁹-Tetrahydrocannabinolic acid (THCA), Δ⁹-Tetrahydrocannabivarin (THCV), (−)-Cannabidivarin (CBDV), Cannabinol (CBN) and Cannabigerol (CBG). Each of these cannabinoids may be present in an amount from about 0.001% to about 40% by weight of the Cannabis extract. Typically, the other cannabinoids are present in amounts lower than the primary cannabinoid or, if present, the secondary cannabinoid.

In some embodiments, certain cannabinoids may be absent, or present in non-detectable amounts (e.g. less than about 0.001% by weight of the analyte). In some embodiments, the Cannabis extract may exclude one or more of the following cannabinoids: Δ⁹-Tetrahydrocannabinolic acid (THCA), Δ⁹-Tetrahydrocannabivarin (THCV), Cannabidiolic acid (CBDA), Cannabinol (CBN), (−)-Cannabidivarin (CBDV), Cannabigerol (CBG) and Cannabichromene (CBC).

Cannabis extracts typically comprise non-cannabinoid compounds, which may include a terpene fraction. In some embodiments, the Cannabis extract comprises a terpene fraction in an amount of less than about 50% by weight, for example, less than about 45%, about 40%, about 35%, about 30%, about 25%, about 20%, about 15%, about 10%, about 9%, about 8%, about 7%, about 6%, about 5%, about 4%, about 3%, about 2% or about 1% by weight of the extract. In some embodiments, the Cannabis extract may comprise terpene and terpenoid compounds in an amount of at least about 0.001% by weight of the extract, for example, at least about 0.005%, about 0.01%, about 0.05%, about 0.1%, about 0.2%, about 0.25%, about 0.3%, about 0.35%, about 0.4%, about 0.45%, about 0.5%, about 1%, about 2%, about 3%, about 4%, about 5%, about 6%, about 10%, about 15% or more of the total weight of the extract. In some embodiments, the Cannabis extract comprises about 0.001% to about 50% by weight of terpene and terpenoid compounds, for example, about 0.01% to about 50% by weight, about 0.01% to about 10% by weight, about 0.01% to about 6% by weight or about 0.01 to about 5% by weight of the pharmaceutical composition.

Typically, the terpene fraction in the plant material used to form the extract may have a different terpene/terpenoid profile than the terpene profile of the final extract, both in terms of the amounts of specific compounds in the terpene fraction and the weight of the terpene fraction relative to the other components. For example, a Cannabis flower may comprise about 20% by weight cannabinoids and about 3% by weight terpenes. Following extraction and concentration (i.e. removal of the extractant), the amount of cannabinoids may increase to an amount of about 50-90% by weight and the terpene fraction may amount to about 0.1-6% by weight of the Cannabis extract. This typical scenario shows that while the cannabinoids are concentrated when the extractant is removed, the relative amount of the terpene fraction is reduced, likely due to the volatility of many of the terpenes/terpenoids present in the terpene fraction. Therefore, the profile of the terpene fraction present in the Cannabis extract is significantly different from the profile of the terpene fraction that exists in Nature.

The efficacy of a composition may be enhanced when the terpene fraction has a certain profile, i.e. a certain proportion of particular terpenes/terpenoids are present in the extract. It is believed that the increase in efficacy may be synergistic (i.e. non-additive). It is also believed that the presence of specific components in the terpene fraction may enhance the patient's tolerance to cannabinoid therapy.

A variety of terpenes and terpenoids have also been identified in Cannabis extracts, including monoterpenes, monoterpenoids, sesquiterpenes and sesquiterpenoids. For example, the following terpenes and terpenoids have been identified in Cannabis extracts: Alloaromadendrene, allyl hexanoate, benzaldehyde, (Z)-a-cis-bergamotene, (Z)-a-trans-bergamotene, ß-bisabolol, epi-a-bisabolol, ß-bisabolene, borneol (camphol), cis-y-bisabolene, bomeol acetate (bomyl acetate), α-cadinene, camphene, camphor, cis-carveol, caryophyllene (ß-caryophyllene), α-humulene (α-caryophyllene), γ-cadinene, Δ-3-carene, caryophyllene oxide, 1,8-cineole, citral A, citral B, cinnameldehyde, α-copaene (aglaiene), γ-curcumene, ß-cymene, ß-elemene, γ-elemene, ethyl decdienoate, ethyl maltol, ethyl propionate, ethylvanillin, eucalyptol, α-eudesmol, ß-eudesmol, γ-eudesmol, eugenol, cis-ß-famesene ((Z)-ß-farnesene), trans-α-farnesene, trans-ß-famesene, trans-γ-bisabolene, fenchone, fenchol (norbomanol, ß-fenchol), geraniol, α-guaiene, guaiol, methyl anthranilate, methyl salicylate, 2-methyl-4-heptanone, 3-methyl-4-heptanone, hexyl acetate, ipsdienol, isoamyl acetate, lemenol, limonene, d-limonene (limonene), linolool (linalyl alcohol, ß-linolool), α-longipinene, menthol, γ-muurolene, myrcene (ß-myrcene), nerolidol, trans-nerolidol, nerol, ß-ocimene (cis-ocimene), octyl acetate, α-phellandrene, phytol, α-pinene (2-pinene), ß-pinene, pulegone, sabinene, cis-sabinene hydrate (cis-thujanol), ß-selinene, α-selinene, γ-terpinene, terpinolene (isoterpine), terpineol (α-terpineol), terpineol-4-ol, α-terpinene (terpilene), α-thujene (origanene), vanillin, viridiflorene (ledene), and α-ylange. In some embodiments, the pharmaceutical composition comprises one or more of these terpenes and/or terpenoids.

In some embodiments, specific terpenes or terpenoids may be absent, or present in non-detectable amounts (e.g. less than about 0.001% by weight of the analyte).

One exemplary Cannabis extract is set out in table 1 below. Amounts of cannabinoids are reported as determined by high-performance liquid chromatography (HPLC), including ultra performance liquid chromatography (UPLC), and amounts of terpenes are reported as determined by HPLC and/or gas chromatography (GC). In some embodiments, the amount of a cannabinoid and/or a terpene may be determined by UPLC using a Waters Acquity UPLC system equipped with a Waters photodiode array detector (PDA) or detection by mass spectrometry. Using UPLC the limit of quantitation (LoQ) of THC, CBD and/or CBN or related substances may be less than 1 μg/ml, for example, the LoQ of CBD may be ≤0.086 μg/ml, CBN may be ≤0.038 μg/ml and/or THC may be ≤0.089 μg/ml may be detected in an analyte. Accordingly, in some embodiments, the pharmaceutical compositions comprise CBD in an amount greater than 0.086 μg/ml and THC in an amount greater than 0.089 μg/ml. It will be appreciated that, as for all plant extracts, the amount of each component may vary in some cases by +/−10%, +/−25% or +/−50%. The ranges of amounts corresponding to each of these limits to account for the potential variation in the composition are also shown in table 1.

TABLE 1

| | Amount | | | |
|---|---|---|---|---|
| | (wt % of | | | |
| | total com- | | | |
| Compound | position) | +/−10% | +/−25% | +/−50% |
| THCA | 0.345 | 0.311-0.380 | 0.259-0.431 | 0.1725- |
| | | | | 0.5175 |
| THC | 55.131 | 49.618-60.644 | 41.348-68.914 | 27.5655- |
| | | | | 82.6965 |
| THCV | Not | | | |
| | detected | | | |
| | (ND) | | | |
| CBD | ND | | | |
| CBDA | ND | | | |
| CBG | 0.367 | 0.330-0.404 | 0.275-0.459 | 0.1835- |
| | | | | 0.5505 |
| CBN | ND | | | |
| CBC | ND | | | |
| α-bisabolol | 0.018 | 0.016-0.020 | 0.0135-0.0225 | 0.009-0.027 |
| camphene | ND | | | |
| δ-s-carene | ND | | | |
| ß- | 0.195 | 0.176-0.215 | 0.146-0.244 | 0.0975- |
| caryophyllene | | | | 0.2925 |
| caryophyllene | 0.003 | 0.0027-0.0033 | 0.00225- | 0.0015- |
| oxide | | | 0.00375 | 0.0045 |
| p-cymene | 0.018 | 0.0162-0.0198 | 0.0135-0.0225 | 0.009-0.027 |
| geraniol | ND | | | |
| guaiol | ND | | | |
| α-humulene | 0.056 | 0.0504-0.0616 | 0.042-0.070 | 0.028-0.084 |
| isopulegol | 0.002 | 0.0018-0.0022 | 0.0015-0.0025 | 0.001-0.003 |
| D-limonene | ND | | | |
| linalool | 0.062 | 0.056-0.068 | 0.047-0.078 | 0.031-0.093 |
| ß-myrcene | 0.002 | 0.0018-0.0022 | 0.0015-0.0025 | 0.001-0.003 |
| nerolidol 1 | 0.036 | 0.032-0.040 | 0.027-0.045 | 0.018-0.054 |
| nerolidol 2 | 0.008 | 0.0072-0.0088 | 0.006-0.010 | 0.004-0.012 |
| ocimene | 0.005 | 0.0045-0.0055 | 0.00375- | 0.0025- |
| | | | 0.00625 | 0.0075 |
| α-pinene | 0.001 | 0.0009-0.0011 | 0.00075- | 0.0005- |
| | | | 0.00125 | 0.0015 |
| ß-pinene | 0.032 | 0.0288-0.0352 | 0.024-0.04 | 0.016-0.048 |
| α-terpinene | 0.001 | 0.0009-0.0011 | 0.00075- | 0.0005- |
| | | | 0.00125 | 0.0015 |
| γ-terpinene | 0.001 | 0.0009-0.0011 | 0.00075- | 0.0005- |
| | | | 0.00125 | 0.0015 |
| terpinolene | 0.002 | 0.0018-0.0022 | 0.0015-0.0025 | 0.001-0.003 |
| Mass (terpene | 0.442 | 0.3978-0.4862 | 0.3315-0.5525 | 0.221-0.663 |
| fraction) | | | | |
| Mass (% of | 56.285 | 50.657-61.914 | 42.214-70.356 | 28.143- |
| total extract) | | | | 84.428 |

The pharmaceutical composition comprises CBD and THC. In some embodiments, CBD and THC are the sole active ingredients in the composition. In some embodiments, the pharmaceutical composition may consist only of CBD and THC. For example, the pharmaceutical composition may be absent any pharmaceutically acceptable excipients, such as a carrier. In some embodiments, the pharmaceutical composition consists essentially of CBD and THC, for example, comprising in addition only minor (e.g. less than 1 wt % relative to the amount of active ingredient(s)) impurities from extraction of the CBD and/or THC from Cannabis. In some embodiments, the pharmaceutical composition consists of a Cannabis extract, for example, a macerated oil, resin or alcoholic extract, optionally with one or more excipients such as a carrier.

In some embodiments, the pharmaceutical composition is in the form of a liquid. References to “liquid” forms of the compositions and/or excipients are intended to refer to compositions that are liquid at 25° C. at 1atm. Typically liquid compositions comprise at least one liquid molecule that is preferably able to solubilise non-liquid molecules present in the composition. Liquid pharmaceutical compositions are advantageous for dosing to subjects (such as young children) unable to reliably swallow solid dosage forms. The ability to accurately administer the correct dosage of the pharmaceutical composition is especially important for treatment of autism and its symptoms since Autism Spectrum Disorder is commonly diagnosed early in life. It will be appreciated that liquid pharmaceutical compositions will comprise a liquid excipient that is typically able to solubilise or suspend the active ingredients. Any liquid excipient disclosed herein may be included in liquid forms of the pharmaceutical compositions, including any of the liquid extractants. Suitable carriers include an alcohol, olive oil, hemp oil, sesame oil, liquid coconut oil, vegetable oil, canola oil, grape seed oil, almond oil, medium-chain triglyceride (MCT) oil, or a combination thereof. In some embodiments, the pharmaceutical composition comprises an alcohol and an MCT oil. In embodiments comprising a Cannabis extract, some particulate material may be present even in embodiments that are in the liquid form since these particulates do not typically contribute to the efficacy of the active ingredients, such as CBD and THC. Liquid pharmaceutical compositions may be suitable for oral, sublingual, parenteral and topical administration.

In some embodiments, the pharmaceutical composition optionally comprises one or more pharmaceutically acceptable excipient(s). The excipient may be a carrier, diluent, adjuvant, or other excipient, or any combination thereof, and “pharmaceutically acceptable” meaning that they are compatible with the other ingredients of the pharmaceutical composition and are not deleterious to a patient upon or following administration. The pharmaceutical compositions may be formulated, for example, by employing conventional solid or liquid vehicles or diluents, as well as pharmaceutical additives of a type appropriate to the mode of desired administration (for example, excipients, binders, preservatives, stabilisers, flavours, etc.) according to techniques such as those well known in the art of pharmaceutical formulation (See, for example, Remington: The Science and Practice of Pharmacy, 21st Ed., 2005, Lippincott Williams & Wilkins). The pharmaceutically acceptable carrier may be any carrier included in the United States Pharmacopeia/National Formulary (USP/NF), the British Pharmacopoeia (BP), the European Pharmacopoeia (EP), or the Japanese Pharmacopoeia (JP). In some embodiments, the excipient may be non-natural (e.g. synthetically produced).

The pharmaceutical composition includes those suitable for oral, rectal, nasal, topical (including buccal and sublingual), vaginal or parenteral (including intramuscular, subcutaneous and intravenous) administration or in a form suitable for administration by inhalation or insufflation. As autism diagnosis often occurs in young children (typically at ages 18-24 months) preferred routes of administration include those suitable for such patients, such as sublingual administration.

The ingredients of the pharmaceutical composition may be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use, in the form of suppositories for rectal administration; or in the form of sterile injectable solutions for parenteral (including subcutaneous) use.

Such pharmaceutical compositions and unit dosage forms thereof may comprise conventional ingredients in conventional proportions, with or without additional active ingredient(s), and such unit dosage forms may contain any suitable effective amount of the active ingredients commensurate with the intended daily dosage range to be employed.

For preparing pharmaceutical compositions described herein, pharmaceutically acceptable carriers can be either solid or liquid. Solid form preparations include powders, tablets, pills, capsules, cachets, suppositories, and dispensable granules. A solid carrier can be one or more substances which may also act as diluents, flavouring agents, solubilisers, lubricants, suspending agents, binders, preservatives, tablet disintegrating agents, or an encapsulating material.

Suitable carriers include magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Tablets, powders, capsules, pills, cachets, and lozenges can be used as solid forms suitable for oral administration.

Liquid form preparations include solutions, dispersions, suspensions, and emulsions, for example, water or water-propylene glycol solutions. For example, parenteral injection liquid preparations can be formulated as solutions in aqueous polyethylene glycol solution. Liquid preparations are preferred for embodiments involving sublingual administration.

In some embodiments, the pharmaceutical composition is formulated for sublingual administration. Therefore, in some embodiments, the pharmaceutical composition is a sublingual pharmaceutical composition. Typically a sublingual pharmaceutical composition is a liquid; however, any other suitable dosage form known in the art may be employed including aerosols, lozenges, troches, films, foams, pastes and dissolvable tablets. In some embodiments, the Cannabis extract is itself in a form suitable for sublingual administration such as a macerated oil or alcoholic extract, and may be used without further formulation. In some embodiments, the Cannabis extract is further formulated to provide the sublingual dosage form.

In some embodiments, the Cannabis extracts that are obtained in the form of resins may be formulated for administration in either a diluted form (e.g. to be administered as sublingual drops) where an edible oil, such as coconut oil, olive oil or sunflower oil, is added to the resin, or in a concentrated form where small quantities of resin (e.g. a quantity of resin approximating the size of a grain of rice, sometimes referred to as the “rice grain” method of administration) are directly administered to the patient.

Sterile liquid form pharmaceutical compositions include sterile solutions, suspensions, emulsions, syrups and elixirs. The active ingredient(s) may be suspended in a pharmaceutically acceptable carrier, such as sterile water, sterile organic solvent or a mixture of both.

Other liquid form preparations include those prepared by combining the Cannabis extract with one or more naturally derived oils (e.g. an essential oil) or waxes. An “essential oil” is an oil derived by extraction (e.g. steam extraction, or contacting the plant material with an extractant) or pressing, which contains primarily hydrophobic, and generally fragrant, components of the plant material. Suitable naturally derived oils and waxes include Sesame oil, Olive oil, Arnica essential oil, Lavender essential oil, Lavender Spike essential oil, Frankincense essential oil, Lemongrass essential oil, Cinnamon Leaf essential oil, Rosemary Cineole essential oil, Rosemary essential oil, Bergamot essential oil, Myrrh essential oil, Sage essential oil, Coconut oil, Bees wax and Hemp oil.

The pharmaceutical compositions may be formulated for parenteral administration (e. g. by injection, for example bolus injection or continuous infusion) and may be presented in unit dose form in ampoules, pre-filled syringes, small volume infusion or in multi-dose containers optionally with an added preservative. The pharmaceutical compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilisation from solution, for constitution with a suitable vehicle, e.g. sterile, pyrogen-free water, before use.

Pharmaceutical forms suitable for injectable use include sterile injectable solutions or dispersions, and sterile powders for the extemporaneous preparation of sterile injectable solutions. They should be stable under the conditions of manufacture and storage and may be preserved against oxidation and the contaminating action of microorganisms such as bacteria or fungi.

The solvent or dispersion medium for the injectable solution or dispersion may contain any of the conventional solvent or carrier systems, and may contain, for example, water, ethanol, polyol (for example, glycerol, propylene glycol and liquid polyethylene glycol, and the like), suitable mixtures thereof, and vegetable oils.

Pharmaceutical forms suitable for injectable use may be delivered by any appropriate route including intravenous, intramuscular, intracerebral, intrathecal, epidural injection or infusion.

Sterile injectable solutions are prepared by incorporating the active ingredients in the required amount in the appropriate carrier with various other ingredients such as those enumerated above, as required, followed by sterilisation. Generally, dispersions are prepared by incorporating the various sterilised active ingredients into a sterile vehicle which contains the basic dispersion medium and the required other ingredients from those enumerated above. In the case of sterile powders for the preparation of sterile injectable solutions, preferred methods of preparation are vacuum drying or freeze-drying of a previously sterile suspension of the active ingredient plus any additional desired ingredients.

For oral administration, the active ingredient(s) may be incorporated with excipients and used in the form of ingestible tablets, buccal tablets, troches, capsules, elixirs, suspensions, syrups, wafers, and the like.

The amount of active ingredient(s) in a therapeutically useful pharmaceutical composition should be sufficient that a suitable dosage will be obtained. Accordingly, the active ingredient(s) are preferably provided in an effective amount.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavouring agent such as peppermint, oil of wintergreen, or cherry flavouring. When the dosage unit form is a capsule, it may contain, in addition to materials of the above type, a liquid carrier.

Various other materials may be present as coatings or to otherwise modify the physical form of the dosage unit. For instance, tablets, pills, or capsules may be coated with shellac, sugar or both. A syrup or elixir may contain the active ingredient(s), sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active ingredient(s) may be incorporated into sustained-release preparations and formulations, including those that allow specific delivery of the active peptide to specific regions of the gut.

Aqueous solutions can be prepared by dissolving the active ingredient(s) in water and adding suitable colorants, flavours, stabilising and thickening agents, as desired. Aqueous suspensions can be made by dispersing the finely divided active ingredient(s) in water with viscous material, such as natural or synthetic gums, resins, methylcellulose, sodium carboxymethylcellulose, or other well-known suspending agents.

Pharmaceutically acceptable carriers and/or diluents include any and all solvents, dispersion media, coatings, antibacterial and antifungal agents, isotonic and absorption delaying agents and the like.

Also included are solid form preparations that are intended to be converted, shortly before use, to liquid form preparations for oral and/or sublingual administration. Such liquid forms include solutions, suspensions, and emulsions. These preparations may contain, in addition to the active ingredient(s), colorants, flavours, stabilisers, buffers, artificial and natural sweeteners, dispersants, thickeners, solubilising agents, and the like.

For topical administration to the epidermis the active ingredient(s) may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Formulations suitable for topical administration in the mouth (e.g. sublingual administration) include any liquid formulation described herein, preferably liquid formulations with a viscosity suitable for administration by dropper or syringe; lozenges comprising active ingredient(s) in a flavoured base, usually sucrose and acacia or tragacanth; pastilles comprising the active ingredient(s) in an inert base such as gelatin and glycerin or sucrose and acacia; and mouthwashes comprising the active ingredient(s) in a suitable liquid carrier.

For administration to the nasal cavity, solutions or suspensions may be applied directly to the nasal cavity by conventional means, for example with a dropper, pipette or spray. The formulations may be provided in single or multidose form. In the latter case of a dropper or pipette, this may be achieved by the patient administering an appropriate, predetermined volume of the solution or suspension.

In the case of a spray, this may be achieved for example by means of a metering atomising spray pump. For such sprays, active ingredient(s) may be encapsulated with cyclodextrins, or formulated with other agents expected to enhance delivery and retention in the nasal mucosa.

Administration to the respiratory tract may be achieved by means of an aerosol formulation in which the active ingredient(s) are provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas.

The aerosol may conveniently also contain a surfactant. The dose of drug may be controlled by provision of a metered valve.

Alternatively the active ingredient(s) may be provided in the form of a dry powder, for example a powder mix of the active ingredient(s) in a suitable powder base such as lactose, starch, starch derivatives such as hydroxypropylmethyl cellulose and polyvinylpyrrolidone (PVP). The pharmaceutical composition as a powder may be presented in unit dose form for example in capsules or cartridges of, e.g. gelatin, or blister packs from which the powder may be administered by means of an inhaler.

In formulations intended for administration to the respiratory tract, including intranasal formulations, the pharmaceutical composition may have a small particle size for example of the order of 5 to 10 microns or less. Such a particle size may be obtained by means known in the art, for example by micronisation.

When desired, formulations adapted to give sustained release of the active ingredient(s) may be employed.

The pharmaceutical composition may be prepared in unit dosage form. In such form the composition is subdivided into unit doses containing appropriate quantities of the active ingredient(s). The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Pharmaceutical compositions for parenteral administration may also be provided in unit dosage form for ease of administration and uniformity of dosage. Unit dosage form as used herein refers to physically discrete units suited as unitary dosages for the patients to be treated; each unit containing a predetermined quantity of active material calculated to produce the desired therapeutic effect in association with the required pharmaceutical excipient. The specification for the unit dosage forms are dictated by and directly dependent on (a) the unique characteristics of the active ingredient(s) and the particular therapeutic effect to be achieved, and (b) the limitations inherent in the art of compounding such an active ingredient(s) for the treatment of living patients having a diseased condition in which bodily health is impaired.

In some embodiments, the pharmaceutical composition further comprises an active ingredient other than CBD and THC. In some embodiments, the pharmaceutical composition further comprises an active ingredient other than a cannabinoid. Any suitable active ingredient may be used provided that the activity of the active ingredient, CBD and/or THC is not diminished when combined. Preferably, the active ingredient is an antipsychotic drug or hormone therapy. In some embodiments, the active ingredient may be any agent described in LeClerc, S. and Easley, D. Pharmacy and Therapeutics 2015; 40(6): 389-397 which is a recent review of pharmacological therapies for ASD. Suitable drugs include risperidone, aripiprazole, clozapine, haloperidol, sertraline, oxytocin, secretin, quetiapine, methylphenidate, venlafaxine, fluoxetine, citalopram, escitalopram, bumetanide, memantine, rivastigmine, mirtazapine, melatonin, acamprosate, atomoxetine, intrathecal baclofen, DMXB-A, vincerinone, RG7314 or a combination thereof. In some embodiments, the patient has previously been administered, or is currently being administered, an antipsychotic drug.

The practice of the present invention employs, unless otherwise indicated, conventional pharmaceutical, veterinary and medical techniques within the skill of the art. Such techniques are well known to the skilled worker, and are explained fully in the literature.

### Methods of Treatment

The present invention also provides a method for treating autism comprising administering to a patient in need thereof an effective amount of a pharmaceutical composition of the invention. Any pharmaceutical composition described herein may be used in this method.

Surprisingly, the inventors observed that this method is able to achieve an improvement in patients diagnosed with autism as assessed by CGI-I. The method may provide treatment of at least one symptom of autism or symptom associated with autism in the patient as assessed by CGI-I. The at least one symptom may be selected from reduced sociability, tantrums, poor use of language, repetitive behaviour, self-injurious behaviour, irritability, hyperactivity, poor ability to focus, unexplained weight loss, fever, fatigue, pain, and skin changes or a combination thereof. In some embodiments, the present invention relates to treating one or more of communication problems; difficulty relating to people, things and events; and repetitive body movements or behaviours in the patient.

As used herein, the term “autism” relates to autism spectrum disorder (ASD) or social (pragmatic) communication disorder (SCD) as defined in the fifth edition of the Diagnostic and Statistical Manual of Mental Disorders (DSM-5) published by the American Psychiatric Association. Typical characteristics of ASD relate to three main categories: communication problems; difficulty relating to people, things and events; and repetitive body movements or behaviours. SCD is a similar disorder to ASD with many overlapping symptoms and behaviours, but sufferers typically do not suffer from restricted and/or repetitive behaviours.

Symptoms of autism include diminished visual contact with others, reduced sociability, tantrums, poor use of language, repetitive behaviour, self-injurious behaviour, irritability, hyperactivity, and poor ability to focus. Symptoms associated with autism include unexplained weight loss, fever, fatigue, pain, and skin changes. In some embodiments, the present invention relates to treating one or more symptoms selected from reduced sociability, tantrums, poor use of language, repetitive behaviour, self-injurious behaviour, irritability, hyperactivity, poor ability to focus, unexplained weight loss, fever, fatigue, pain, and skin changes or a combination thereof. In some embodiments, the present invention relates to treating one or more of communication problems; difficulty relating to people, things and events; and repetitive body movements or behaviours in the patient.

One system for assessing the severity of autism is the Clinical Global Impressions (CGI) scale. The CGI scale is described in Busner, J. and Targum, S. D. Psychiatry (Edgmont) 2007; 4(7): 28-37. The CGI scale has two components CGI-Severity (CGI-S) and CGI-Improvement (CGI-I). CGI-S rates the average severity of the patient's mental health over the preceding 7-day period on a 7-point scale, while CGI-I rates the improvement of the patient using the same metrics as CGI-S after commencement of a treatment. CGI is assessed by a clinician or experienced researcher.

As used herein, the terms “treating”, “treatment”, “treat” and the like mean affecting a subject, patient, tissue or cell to obtain a desired pharmacological and/or physiological effect. The effect may be prophylactic in terms of completely or partially preventing, or reducing the severity of, a disease or associated symptom, and/or may be therapeutic in terms of a partial or complete cure of a disease. A reference to “treating” autism therefore encompasses: (a) arresting the progress of the disease, e.g. preventing worsening of a symptom over time as assessed by the CGI-I; (b) relieving or ameliorating the effects of autism, i.e. causing an improvement of at least one symptom of autism as assessed by the CGI-I; (c) preventing additional symptoms from developing; or (d) preventing autism or symptom associated with autism from occurring in a patient predisposed to autism or at risk thereof so that autism does not develop or occur in the patient. In some embodiments, the invention provides a method of arresting the progress of autism. In some embodiments, the invention provides a method of improving at least one symptom of autism as assessed by the CGI-I. In some embodiments, the invention provides a method of preventing additional symptoms of autism from developing as assessed by the CGI scale. In some embodiments, the invention provides a method of preventing autism or symptom associated with autism from occurring in a patient predisposed to autism or at risk thereof so that autism does not develop or occur in the patient.

The term “administering” refers to providing the pharmaceutical composition to a patient suffering from or at risk of the disease(s) or condition(s) to be treated or prevented.

By “effective amount” it is meant an amount sufficient that, when administered to the patient, an amount of the drug is provided to achieve an effect. In the case of a therapeutic method, this effect may be the treatment of autism or a symptom associated with autism. Therefore, the “effective amount” may be a “therapeutically effective amount”. By “therapeutically effective amount” it is meant an amount sufficient that when administered to the patient an amount of active ingredient is provided to treat the disease or a symptom of the disease.

The method may comprise administering CBD and/or THC in a low dose. By administering the active ingredient(s) in a low dose, the frequency and/or severity of adverse events resulting from treatment may be reduced. A previously described cannabinoid therapy involves dosages of CBD or THC of up to 500 mg. For treating autism, low doses of CBD may comprise administering less than about 20 mg/day, for example, less than about 19, about 18, about 17, about 16, about 15 or about 14 mg/day. In some embodiments, CBD may be administered in an amount from about 0.1 mg/day to about 20 mg/day, about 0.1 mg/day to about 18 mg/day or about 0.1 mg/day to about 15 mg/day. Low doses of THC for treating autism may comprise administering less than about 10 mg/day, for example, less than about 9, about 8, about 7 mg/day or about 6 mg/day. Thus, in some embodiments, THC may be administered in an amount from about 0.1 mg/day to about 10 mg/day, about 0.1 mg/day to about 8 mg/day or about 0.15 mg/day to about 6 mg/day. Any of these dosage values for CBD may be combined with dosage values for THC provided the amounts selected provide the desired ratio of CBD:THC in the pharmaceutical composition.

In some embodiments, the method comprises administering a total dose of cannabinoids (e.g. THC, CBD and any other cannabinoid(s) present) of less than 30 mg/day, for example, from about 0.1 to about 30 mg/day or from about 0.1 to about 15 mg/day. In some embodiments, the combined dose of CBD and THC may be less than about 30 mg/day, for example, less than about 25, about 20, about 15, about 10 or about 5 mg/day. In some embodiments, the dose of CBD and THC combined may be from about 0.1 mg/day to about 30 mg/day, about 0.1 mg/day to about 20 mg/day or about 0.1 mg/day to about 15 mg/day.

In some embodiments, the pharmaceutical composition may be administered 1, 2, 3, 4 or more times per day, preferably the pharmaceutical composition is administered twice daily. In order to facilitate the desired dosing schedule, the pharmaceutical composition may be formulated with a convenient concentration of active ingredient(s). For example, in the case of a pharmaceutical composition intended for sublingual administration, in some embodiments, the pharmaceutical composition may be formulated to administer THC in about 0.01 mg/drop to about 10 mg/drop, for example, about 0.05 mg/drop to about 8 mg/drop or about 0.1 mg/drop to about 1 mg/drop. In these embodiments, the pharmaceutical composition may be formulated to administer CBD in about 0.01 mg/drop to about 10 mg/drop, for example, about 0.05 mg/drop to about 8 mg/drop or about 0.1 mg/drop to about 1 mg/drop. Therefore, in one embodiment, a pharmaceutical composition may comprise about 1 mg to about 100 mg THC and about 1 mg to about 100 mg CBD in a solution of about 100 ml. The person skilled in the art will readily be able to prepare any volume solution to provide the desired dosage and desired relative amount of active ingredient(s).

The treatment may be maintained over an extended period of time. Maintenance of treatment includes continual or periodic treatment according to the method described herein. For example, in some embodiments, the treatment should be maintained continuously for at least 4, 6, 8, 10, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 or more weeks. Typically the treatment is continued for at least 12 weeks with administration at least once daily, preferably twice daily.

The patient may be recently diagnosed with autism or may have been previously treated with existing therapies for autism. For example, in some embodiments, the patient may have previously been treated with an existing therapy, such as risperidone, aripiprazole, quetiapine and/or methylphenidate. For patients previously treated, the previous treatment may have been successful, moderately successful or not successful as assessed by CGI-I. Advantageously, treatment according to the methods described herein may be effective for patients who did not respond to an existing therapy, such as risperidone, aripiprazole, quetiapine and/or methylphenidate therapy.

The method may comprise administering more than one pharmaceutical composition of the present invention to the patient in need thereof. For example, in some circumstances, it is preferred to administer a pharmaceutical composition high in THC and a pharmaceutical composition high in CBD to the patient. These pharmaceutical compositions may be administered in an alternating order and separated by a period of time. For example, the administration of high-THC and high-CBD formulations may be on alternating days, alternating sequences of days, or alternating from a high-CBD formulation in the morning to a high-THC formulation at night.

The method may also comprise administering an active ingredient other than CBD and/or THC. This active ingredient may be administered simultaneously, separately or consecutively with the CBD and/or THC. By simultaneously it is meant that each of pharmaceutical composition and the other active ingredient are administered at the same time either in the same pharmaceutical composition. By separately it is mean that each of pharmaceutical composition and the other active ingredient are administered at the same time in different pharmaceutical compositions and optionally by different routes of administration. By consecutively it is meant that each of pharmaceutical composition and the other active ingredient are administered separately and may be at different times. Typically, when the pharmaceutical composition and the other active ingredient are administered consecutively they are administered within 24 hours, or within 12, 8, 6, 5, 4, 3, 2, or 1 hour(s) of each other. The pharmaceutical composition may be administered before or after the other active ingredient. Further, the route of administration for the pharmaceutical composition and the other active ingredient may be the same or different. In some embodiments, the other active ingredient may be any existing therapy for autism, such as administration of risperidone, aripiprazole, clozapine, haloperidol, sertraline, oxytocin, secretin, quetiapine, methylphenidate, venlafaxine, fluoxetine, citalopram, escitalopram, bumetanide, memantine, rivastigmine, mirtazapine, melatonin, acamprosate, atomoxetine, intrathecal baclofen, DMXB-A, vincerinone, RG7314 or a combination thereof.

The pharmaceutical composition may be administered by any suitable route of administration. In some embodiments, the pharmaceutical composition is administered sublingually. Sublingual dosing is advantageous in particular to very young patients at the early stages of autism diagnosis. To facilitate sublingual administration the pharmaceutical composition is typically a liquid. Typically sublingual administration is achieved by a medical device, such as a dropper or syringe.

The present invention also provides use of CBD and THC in a ratio of CBD:THC from about 0.1:5 to about 5:0.1 in the manufacture of a medicament for treating autism. Also provided is the use of CBD in the manufacture of a medicament for treating autism, wherein the medicament comprises CBD and THC in a ratio of CBD:THC from about 0.1:5 to about 5:0.1. Also provided is the use of THC in the manufacture of a medicament for treating autism, wherein the medicament comprises CBD and THC in a ratio of CBD:THC from about 0.1:5 to about 5:0.1. Also provided is the use of a Cannabis extract in the manufacture of a medicament for treating autism, wherein the medicament comprises CBD and THC in a ratio of CBD:THC from about 0.1:5 to about 5:0.1. The medicament may be the same as the pharmaceutical compositions described herein, including comprising any of the ratios of CBD:THC, excipients or other molecules described herein.

The present invention further provides a kit comprising in separate parts: (a) CBD and (b) THC, wherein the amount of CBD in part (a) and the amount of THC in part (b) are in a ratio of CBD:THC from about 0.1:5 to about 5:0.1. Preferably, the kit comprises CBD and/or THC in an effective amount.

In some embodiments, part (a) and/or part (b) of the kit further comprise a pharmaceutically acceptable excipient. In other embodiments, the pharmaceutically acceptable excipient is provided in a further part of the kit, part (c).

In some embodiments, the kit may comprise in a separate part (d) an active ingredient other than CBD and/or THC. Part (d) may be included in the kit, in addition to parts (a), (b) and/or (c).

In some embodiments, the kit may comprise in a separate part (e) a medical device, such as a dropper or syringe, and optionally (f) instructions for its use. Parts (e) and (f) may be independently included in the kit in addition to parts (a), (b), (c) and/or (d).

In some embodiments, parts (a), (b) and (c) of the kit may be combined to provide any of the pharmaceutical compositions described herein.

Also described herein is a kit comprising in separate parts: i. a pharmaceutical composition described herein, and ii. instructions for its use.

In another aspect, the invention provides an agent comprising CBD and THC in a ratio of CBD:THC from about 0.1:5 to about 5:0.1. The agent may further comprise any of the ingredients of a pharmaceutical composition described herein.

It must be noted that as used herein and in the appended claims, the singular forms “a,” “an,” and “the” include plural reference unless the context clearly dictates otherwise.

Thus, for example, a reference to “an excipient” may include a plurality of excipients, and a reference to “a patient” may be a reference to one or more patients, and so forth. Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

The term “(s)” following a noun contemplates the singular or plural form, or both.

The term “and/or” can mean “and” or “or”.

Unless the context requires otherwise, all percentages referred to herein are percentages by weight of the pharmaceutical composition. Similarly, unless the context requires otherwise, all ratios referred to herein are ratios by weight.

Various features of the invention are described and/or claimed with reference to a certain value, or range of values. These values are intended to relate to the results of the various appropriate measurement techniques, and therefore should be interpreted as including a margin of error inherent in any particular measurement technique. Some of the values referred to herein are denoted by the term “about” to at least in part account for this variability. The term “about”, when used to describe a value, preferably means an amount within ±25%, ±10%, ±5%, ±1% or ±0.1% of that value.

Various values are described in terms of their percentage relative to the total weight of (i) the pharmaceutical composition, (ii) Cannabis extract or (iii) fraction of the extract (e.g. the cannabinoid fraction or the terpene fraction). The percentages of components included in the Cannabis extract or a fraction thereof (e.g. the cannabinoid fraction or terpene fraction) are intended to denote the percentage by weight of the specified compound relative to the percentage by weight of the other compounds present in the extract or specified fraction, for example, absent the carriers, diluents, adjuvants and excipients or any combination thereof. For example, a pharmaceutical composition comprising a Cannabis extract comprising a terpene fraction in an amount of at least 3% by weight of the extract is intended to denote a pharmaceutical composition wherein the cumulative weight of terpenes and terpenoids is 3% by weight or more when compared to the cumulative weight of compounds present in the extract including cannabinoids, terpenes, terpenoids and extractant/residual extractant. Further, a Cannabis extract comprising THC in an amount of about 85% by weight of the cannabinoid fraction is intended to denote an extract comprising THC in an amount of 85% by weight relative to the cumulative weight of all cannabinoids present in the extract.

The term “comprising” as used in this specification means “consisting at least in part of”. When interpreting statements in this specification that include that term, the features, prefaced by that term in each statement, all need to be present but other features can also be present. Related terms such as “comprise” and “comprised” are to be interpreted in the same manner.

### EXAMPLE(S)

The invention will be further described by way of non-limiting example(s). It will be understood to persons skilled in the art of the invention that many modifications may be made without departing from the spirit and scope of the invention.

### Example 1—Preparation of Sublingual Formulations

Sublingual formulations were prepared from Cannabis extracts obtained as macerated oils or resins. Their manufacture is described in this Example.

### Macerated Oil (Sublingual Drops)

Dried Cannabis buds were contacted with olive oil (100 ml oil per 10 g of dried Cannabis buds). The mixture was heated for 3 hours over a water bath (as a double-boiler setup). Then, after the Cannabis oil had cooled, the mixture was filtered to provide the macerated oil.

### Resin

Dried Cannabis buds were contacted with ethanol (99.7%) (1 litre of ethanol per 50 g of dried Cannabis bud). After extraction, the mixture was filtered and heated for approx. 2-4 hours (depending on the heating system) to a maximum of 90° to evaporate the ethanol.

To prepare sublingual drops from the resin, coconut oil was added to the resin until the desired concentration was obtained. Drops were prepared at different concentrations, including: 3 parts resin to 1 part of coconut oil, 0.5 ml resin to 30 ml coconut oil and 1 ml resin to 30 ml coconut oil.

The resin may also be administered in the form of a “rice grain”. This solid sublingual dosage form was prepared by placing a rice grain sized amount of resin (prepared as above) onto an edible vehicle or directly under the tongue.

### Example 2—Treatment of Autism Spectrum Disorder (ASD)

A retrospective review of patients seen between June 2016 and March 2017 with ASD diagnosis according to DSM-5 (persistent deficits in social communication and interaction; restricted, repetitive patterns of behaviour, interests, or activities; symptoms present in the early developmental period; cause clinically significant impairment in social, occupational, or other areas of current functioning; not explained by intellectual disability nor global developmental delay) who were treated with Cannabis extracts for at least a 12-week period was completed. Demographic/clinical data, neuroimaging/EEG studies, vision, audition, genetic, and metabolic tests, parental/school/neuropsychological reports were reviewed.

The patients were administered with the cannabinoids sublingually (sublingual drops or “rice grain” method—see Example 1) twice daily (BID) for the duration of their treatment. The Cannabis extracts were obtained from the Moby Dick, Sharck, Durga mata or Nebula strains of Cannabis. For 10 out of the 21 patients included in the study, the total daily dosage of CBD and THC (measured by HPLC) and CBD:THC ratio were calculated and discussed further below.

The measured dosage of CBD and THC are shown in Table 2.

TABLE 2
Daily administered dose of CBD and THC (n = 10)

| | Range of | Mean administered |
|---|---|---|
| Type of cannabinoid | administered dose | dose |
| Cannabidiol (CBD) | 0.17-13.32 mg/day | 1.94 mg/day |
| Tetrahydrocannabinol (THC) | 0.22-5.26 mg/day | 0.89 mg/day |

The patients were administered THC and CBD in one of the following formulations outlined in Table 3. Formulations 1-7 of Table 3 were prepared by methods corresponding to those described in Example 1.

TABLE 3

| Formu- | | | THC | CBD | |
|---|---|---|---|---|---|
| lation | Cannabis | | concentration | concentration | CBD: |
| ID | strain | Dosage form | (mg/ml) | (mg/ml) | THC |
| 1 | Durga | Sublingual | 0.88 | 2.22 | 2.5 |
| | mata | drops (diluted | | | |
| | | resin) | | | |
| 2 | Durga | Sublingual | 0.44 | 1.11 | 2.5 |
| | mata | drops | | | |
| | | (macerated oil) | | | |
| 3 | Durga | Resin (“rice | 26.28 | 66.58 | 2.5 |
| | mata | grain”) | | | |
| 4 | Nebula | Sublingual | 0.74 | 1.04 | 1.4 |
| | | drops | | | |
| | | (macerated oil) | | | |
| 5 | Nebula | Sublingual | 0.74 | 1.04 | 1.4 |
| | | drops (diluted | | | |
| | | resin) | | | |
| 6 | Moby | Sublingual | 0.47 | 0.21 | 0.45 |
| | dick | drops | | | |
| | | (macerated oil) | | | |
| 7 | Sharck | Sublingual | 0.27 | 0.40 | 1.5 |
| | | drops | | | |
| | | (macerated oil) | | | |

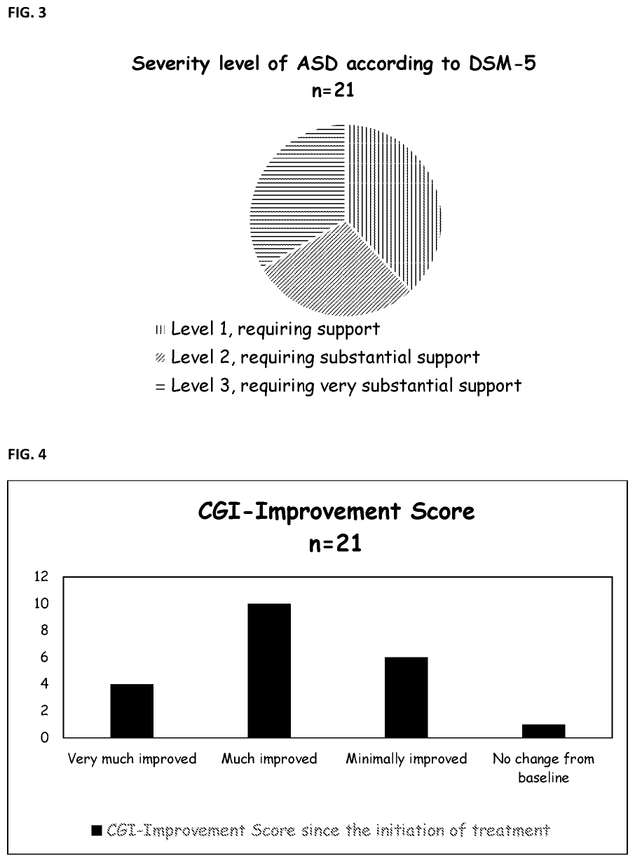

The initial severity of symptoms presenting in the patients was assessed according to the criteria provided in the DSM-5, and are shown in FIG. 3. Clinical response to treatment was estimated using Clinical Global Impression of Improvement (CGI-I) Scale and results are shown in FIG. 4.

The selected patients consisted of twenty (20) children and 1 adult (Mean age: 9 years, 10 month (range: 26 mo-22 yo), 15 males). FIG. 3 shows severity level of ASD at baseline. 66.6% of patients were previously treated with risperidone, aripiprazole, quetiapine and/or methylphenidate, all of them without good response and/or with undesirable adverse events.

Mean follow-up after starting Cannabis therapy was 7.6 mo (range: 3-12 months). According to Cannabis strain, 71.5% of patients received balanced CBD:THC extracts; 19.0% high-CBD (e.g. Table 3; Formulations 1-3), and 9.5% high-THC extracts (e.g. Table 3; Formulation 6). The administered daily dose of CBD and THC was measured in 10 patients; 9 of them received high-CBD and one high-THC extracts (Tables 2 and 3).

According to the CGI-I Scale, 66.65% of patients had significant general improvement (very much improved: 19.0%, much improved: 47.6%) (FIG. 4). 71.4% of cases improved at least one of the core symptoms of ASD, including social communication, language, or repetitive behaviours. Additionally, food acceptance or feeding (6 out of 7 patients), sleep disorders (6 out of 10 patients), sensory difficulties (2 out of 5 patients), and/or seizures (2 out of 3 patients) were improved.

Adverse events included more agitation (two patients), more irritability (one patient), somnolence (one patient), insomnia (one patient), and seizure aggravation (one patient), but they were easily solved by changing the strain. Another patient had constipation.

In this Example, pharmaceutical compositions comprising THC and CBD were dramatically more effective than conventional medicines previously used, and they were well tolerated overall. According to the CGI-I Scale (FIG. 4), 66.65% of patients had significant improvement. Further, 71.4% of cases improved at least one of the core symptoms of ASD, including social communication, language, or repetitive behaviours.

1-29. (canceled)

30. A method for treating autism, an autism spectrum disorder, or a symptom thereof, comprising administering to a patient in need thereof an effective amount of a pharmaceutical composition, wherein the pharmaceutical composition comprises one or more Cannabis extracts, the extract or combined extracts comprising:
cannabidiol (CBD) and Δ⁹-tetrahydrocannabinol (THC) in a weight ratio of CBD:THC from about 1:1 to about 1:5; and
a terpene fraction present in an amount of at least 6% by weight of the Cannabis extract or combined extracts, wherein the terpene fraction comprises one or more terpene or terpenoid compounds selected from the group consisting of: eucalyptol, α-pinene, ß-caryophyllene, and ß-myrcene.

31. The method of claim 30, wherein the pharmaceutical composition comprises CBD:THC in a weight ratio of about 1:1.

32. The method of claim 30, wherein the pharmaceutical composition comprises CBD:THC in a weight ratio of about 1:5.

33. The method of claim 30, wherein the pharmaceutical composition comprises eucalyptol and/or α-pinene.

34. The method of claim 30, wherein the pharmaceutical composition comprises ß-caryophyllene and/or and ß-myrcene.

35. The method of claim 30, wherein the Cannabis extract is a macerated oil or a resin.

36. The method of claim 30, wherein the pharmaceutical composition comprises cannabigerol.

37. The method of claim 30, wherein the pharmaceutical composition comprises a pharmaceutically acceptable carrier selected from the group consisting of olive oil, sesame oil, canola oil, grape seed oil, almond oil, medium-chain triglyceride (MCT) oil, and combinations thereof.

38. The method of claim 37, wherein the pharmaceutical composition comprises olive oil.

39. The method of claim 30, wherein the pharmaceutical composition is in the form of a liquid.

40. The method of claim 30, wherein the pharmaceutical composition is formulated for sublingual administration.

41. The method of claim 30, wherein the method is for treating a symptom selected from the group consisting of reduced sociability, tantrums, poor use of language, repetitive behavior, self-injurious behavior, irritability, hyperactivity, poor ability to focus, unexplained weight loss, fever, fatigue, pain, and skin changes or a combination thereof.

42. The method of claim 30, wherein treatment is maintained for at least about 12 weeks.

43. The method of claim 30, wherein THC is administered in an amount of less than about 10 mg/day.

44. The method of claim 30, wherein THC is administered in an amount from about 0.1-10 mg/day.

45. The method of claim 30, wherein CBD is administered in an amount of less than about 20 mg/day.

46. The method of claim 30, wherein CBD is administered in an amount from about 0.1-20 mg/day.

47. The method of claim 30, wherein the pharmaceutical composition is administered twice daily.

48. The method of claim 30, wherein the pharmaceutical composition is administered sublingually.
